(19) United States
(12) Patent Application Publication
(10) Pub. No.: US 2025/0201353 A1
(43) Pub. Date: Jun. 19, 2025

(54) MOLECULAR SIMULATION METHOD AND INFORMATION PROCESSING APPARATUS

(71) Applicant: Fujitsu Limited, Kawasaki-shi (JP)

(72) Inventors: Yasunari HIKIMA, Kawasaki (JP); Yuhei UMEDA, Kawasaki (JP)

(21) Appl. No.: 19/072,877

(22) Filed: Mar. 6, 2025

Related U.S. Application Data
(63) Continuation of application No. PCT/JP2022/040286, filed on Oct. 28, 2022.

(51) Int. Cl. G16C 10/00; G16C 20/30; G16C 20/70

(52) U.S. Cl. CPC G16C 10/00; G16C 20/30; G16C 20/70

(57) ABSTRACT

An information processing apparatus computes, based on molecular information, for each of a plurality of interatomic distances, an estimated execution time and an estimated number of iterations of an algorithm that computes a molecular energy by using quantum circuit data. The information processing apparatus determines a limited number of iterations for each of the plurality of interatomic distances, based on a time limit and based on the estimated execution time and the estimated number of iterations. The information processing apparatus computes the molecular energy for each of the plurality of interatomic distances based on the limited number of iterations, by executing the algorithm.

### CROSS-REFERENCE TO RELATED APPLICATION

This application is a continuation application of International Application PCT/JP2022/040286 filed on Oct. 28, 2022, which designated the U.S., the entire contents of which are incorporated herein by reference.

### FIELD

The embodiments discussed herein relate to a molecular simulation method and an information processing apparatus.

### BACKGROUND

There are cases in which a computer executes a molecular simulation for analyzing characteristics of a molecule through a numerical computation. There are cases in which the molecular simulation is used in industrial fields such as material development and drug development. Examples of the molecular simulation include a quantum chemical computation for microscopically computing the energy of a molecular based on the electronic state of the molecule and a Schrödinger equation.

For example, the variational quantum eigensolver (VQE) is a quantum chemical computation algorithm that uses quantum circuit data. The algorithm using quantum circuit data may be executed by a quantum computer. For example, configuration interaction (CI) and coupled cluster (CC) are other quantum chemical computation algorithms.

There has been proposed a system in which a quantum state is generated by using control parameters, an energy corresponding to the quantum state is measured, the control parameters are updated such that the energy drops, and this process is iterated until a minimum energy is computed. There has also been proposed a method for determining the energy level of a physical system by using a quantum computer. There has also been proposed an information processing apparatus that searches for the ground-state energy of a molecule by controlling a plurality of quantum computers. See, for example, the following documents.
International Publication Pamphlet No. WO2019/057317
International Publication Pamphlet No. WO2019/150090
International Publication Pamphlet No. WO2022/107298

### SUMMARY

In one aspect, there is provided a non-transitory computer-readable recording medium storing therein a computer program that causes a computer to execute a process including: computing, based on molecular information about an analysis target molecule, for each of a plurality of interatomic distances, an estimated execution time of a first algorithm that computes a molecular energy by using quantum circuit data and an estimated number of iterations of an iteration process executed during the estimated execution time; determining a limited number of iterations for each of the plurality of interatomic distances, based on a specified time limit and based on the estimated execution time and the estimated number of iterations for each of the plurality of interatomic distances; and computing the molecular energy for each of the plurality of interatomic distances based on the limited number of iterations determined, by executing the first algorithm.

The object and advantages of the invention will be realized and attained by means of the elements and combinations particularly pointed out in the claims.

It is to be understood that both the foregoing general description and the following detailed description are exemplary and explanatory and are not restrictive of the invention.

### BRIEF DESCRIPTION OF DRAWINGS

FIG. 1 illustrates an information processing apparatus according to a first embodiment;

FIG. 2 illustrates a hardware example of an information processing apparatus according to a second embodiment;

FIG. 3 illustrates accuracy examples of potential energy curves;

FIG. 4 illustrates an example of the relationship between the interatomic distance and the number of iterations of a classical algorithm;

FIG. 5 illustrates an example of the error of the classical algorithm and an example of the slope of the number of iterations;

FIG. 6 illustrates an example of estimation of an execution time of the variational quantum eigensolver (VQE);

FIG. 7 illustrates a first example of adjustment of VQE execution times;

FIG. 8 illustrates a second example of adjustment of VQE execution times;

FIG. 9 illustrates a third example of adjustment of VQE execution times;

FIG. 10 illustrates accuracy examples of potential energy curves when reduction in execution time is executed;

FIG. 11 illustrates examples of VQE errors;

FIG. 12 is a block diagram illustrating an example of functions of the information processing apparatus;

FIG. 13 is a flowchart illustrating an example of the first half of a procedure of a quantum chemical computation; and

FIG. 14 is a flowchart illustrating an example of the second half of the procedure of the quantum chemical computation.

### DESCRIPTION OF EMBODIMENTS

There are cases in which a computer computes the energy of a molecule while changing the distance between two target atoms and analyzes the relationship between the interatomic distance and the molecular energy. For example, there are cases in which the computer generates a potential energy curve (PEC) indicating the relationship between the interatomic distance and the ground-state energy of the molecule.

However, regarding quantum chemical computation algorithms, there is a trade-off between the accuracy and the execution time. Although an algorithm such as VQE using quantum circuit data is able to compute an accurate molecular energy, the algorithm needs a long execution time. In this connection, it is not always the case that the computer may take much time for the quantum chemical computation, and for example, a time limit may be specified by the user.

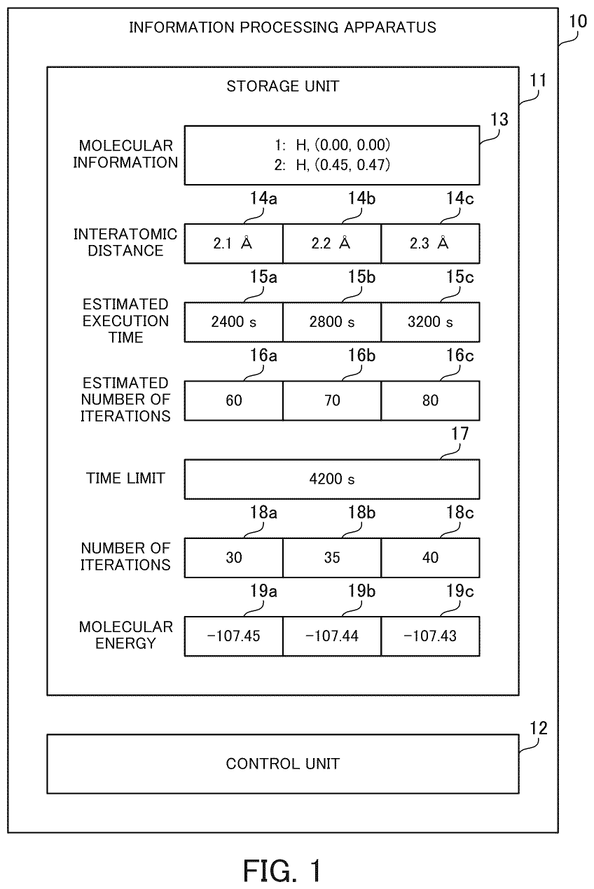

Hereinafter, embodiments will be described with reference to the accompanying drawings. First, a first embodiment will be described. FIG. 1 illustrates an information processing apparatus according to the first embodiment. This information processing apparatus 10 according to the first embodiment executes a molecular simulation based on a quantum chemical computation. The information processing apparatus 10 computes a plurality of molecular energies corresponding to a plurality of interatomic distances, and outputs information in which the interatomic distances and the molecular energies are associated with each other. For example, the information processing apparatus 10 generates and outputs a potential energy curve. The information processing apparatus 10 may be a client apparatus or a server apparatus. The information processing apparatus 10 may be referred to as a computer, a molecular simulation apparatus, or a quantum chemical computation apparatus.

The information processing apparatus 10 includes a storage unit 11 and a control unit 12. The storage unit 11 may be a volatile semiconductor memory such as a random access memory (RAM) or may be a non-volatile storage such as a hard disk drive (HDD) or a flash memory.

The control unit 12 is, for example, a processor such as a central processing unit (CPU), a graphics processing unit (GPU), or a digital signal processor (DSP). The control unit 12 may include an electronic circuit such as an application specific integrated circuit (ASIC) or a field programmable gate array (FPGA). The processor executes a program stored in a memory such as a RAM (the memory may be the storage unit 11), for example. The processor may be referred to as processor circuitry. A group of processors may be referred to as a multiprocessor or simply a “processor”. Of all the plurality of processes to be described below, different processes may be executed by different processors.

The storage unit 11 stores molecular information 13 about an analysis target molecule. The molecular information 13 indicates a molecular structure, such as the kind and coordinates of each of the plurality of atoms included in the molecule. In addition, the storage unit 11 stores a plurality of interatomic distances for which a molecular energy is calculated based on an algorithm. The individual interatomic distance is the distance between two target atoms of the molecule. The distance is, for example, the Euclidean distance. The molecular energy is, for example, the ground-state energy obtained when the molecule is in a stable state. The molecular energy changes as the interatomic distance changes.

For example, the storage unit 11 stores interatomic distances 14a, 14b, and 14c. The interatomic distance 14b is longer than the interatomic distance 14a, and the interatomic distance 14c is longer than the interatomic distance 14b. The storage unit 11 also stores a time limit 17. The time limit 17 is the upper limit of the time for the quantum chemical computation, and may be specified by the user. The time limit 17 is the upper limit of the total execution time for the above-described algorithm to compute all the plurality of molecular energies corresponding to the plurality of interatomic distances stored in the storage unit 11, for example.

The above-described algorithm is for computing a molecular energy by using quantum circuit data. For example, the above-described algorithm is a quantum algorithm such as VQE. The quantum circuit data is a quantum computation model that defines gate operations executed on qubits.

The above-described algorithm may be executed by a gate-based quantum computer. The algorithm may be executed by a von Neumann classical computer by using software that simulates an operation of a quantum computer. The information processing apparatus 10 may execute the algorithm by itself or may causes a different information processing apparatus to execute the algorithm.

For example, the quantum circuit data includes an ansatz circuit and a measurement circuit. The ansatz circuit is for generating a quantum state by using one or more qubits, and is generated based on basis functions that approximate the wave function of a Schradinger equation. The measurement circuit is for measuring a molecular energy from the quantum state, and is generated based on the Hamiltonian of the Schradinger equation, depending on the kind of the molecule.

For example, the above-described algorithm generates a quantum state in a certain electron configuration and measures a molecular energy. The algorithm iterates this process a plurality of times, to compute a molecular energy expectation value in this electron configuration. The algorithm iterates the computation of the molecular energy expectation value while changing the electron configuration, so as to search for a minimum molecular energy. The algorithm outputs the minimum molecular energy as the ground-state energy.

The information processing apparatus 10 may compute other molecular energies corresponding to other interatomic distances by using a different algorithm other than the above-described algorithm. These other interatomic distances may be shorter than the above-described interatomic distances stored in the storage unit 11. The storage unit 11 may also store these other interatomic distances.

The different algorithm computes molecular energies by using a method different from that used by the above-described algorithm. For example, the different algorithm is a classical algorithm that does not use the quantum circuit data, and is assumed to be executed by a classical computer. The different algorithm may be configuration interaction (CI) such as configuration interaction singles and doubles (CISD) or coupled cluster (CC) such as coupled cluster singles and doubles (CCSD) or CCSD and triples (CCSD(T)).

For example, the different algorithm generates a certain computation expression based on basis functions that approximate a wave function, and computes a molecular energy about a certain electron configuration. In this case, for reduction of the computation amount, the different algorithm may ignore higher-order electron excitations such as three-electron excitation or greater or four-electron excitation or greater. The different algorithm searches for a minimum molecular energy by iterating the computation of the molecular energy while changing the electron configuration. The different algorithm outputs the minimum molecular energy as the ground-state energy.

Based on the molecular information 13, the control unit 12 computes estimated execution times 15a, 15b, and 15c and estimated numbers of iterations 16a, 16b, and 16c for the interatomic distances 14a, 14b, and 14c. The estimated execution time 15a and the estimated number of iterations 16a correspond to the interatomic distance 14a. The estimated execution time 15b and the estimated number of iterations 16b correspond to the interatomic distance 14b. The estimated execution time 15c and the estimated number of iterations 16c correspond to the interatomic distance 14c.

Each of the estimated execution times 15a, 15b, and 15c is the time for the algorithm to output a single final molecular energy corresponding to a single interatomic distance. Each of the estimated execution times 15a, 15b, and 15c includes the time for the iteration process executed until the molecular energy converges. Each of the estimated numbers of iterations 16a, 16b, and 16c is the number of iterations of the above-described iteration process. Each of the estimated execution times 15a, 15b, and 15c and the estimated numbers of iterations 16a, 16b, and 16c is an estimated value when no limitation is set on any of the numbers of iterations of the algorithm. For example, each of the estimated execution times 15a, 15b, and 15c is an estimated value of the corresponding maximum execution time, and each of the estimated numbers of iterations 16a, 16b, and 16c is an estimated value of the corresponding maximum number of iterations.

For each of the plurality of interatomic distances, the control unit 12 may generate quantum circuit data from the molecular information 13, and may compute an estimated execution time by using the features of the structure of the corresponding quantum circuit data. In addition, for each of the plurality of interatomic distances, the control unit 12 may execute the different algorithm such as CI or CC, and may compute an estimated number of iterations based on the execution result of the different algorithm. The execution result of the different algorithm may be a measured value of the corresponding number of iterations of the iteration process executed by the different algorithm.

The control unit 12 determines the numbers of iterations 18a, 18b, and 18c for the interatomic distances 14a, 14b, and 14c, based on the time limit 17, the estimated execution times 15a, 15b, and 15c, and the estimated numbers of iterations 16a, 16b, and 16c. The number of iterations 18a corresponds to the interatomic distance 14a, the number of iterations 18b corresponds to the interatomic distance 14b, and the number of iterations 18c corresponds to the interatomic distance 14c.

For example, the numbers of iterations 18a, 18b, and 18c are each the upper limit of the corresponding number of iterations of the above-described algorithm, and each correspond to a limited number of iterations. The numbers of iterations 18a, 18b, and 18c may be equal to or less than their respective estimated numbers of iterations, and at least one of the numbers of iterations 18a, 18b, and 18c may be less than its corresponding estimated number of iterations. For example, if the sum of the estimated execution times 15a, 15b, and 15c is equal to or less than the time limit 17, the control unit 12 does not limit the number of iterations for any of the interatomic distances. However, if the sum of the estimated execution times 15a, 15b, and 15c exceeds the time limit 17, the control unit 12 limits the number of iterations for at least one of the interatomic distances.

For example, the control unit 12 computes a ratio from the sum of the estimated execution times 15a, 15b, and 15c and the time limit 17, and multiplies the computed ratio by the estimated numbers of iterations 16a, 16b, and 16c, so as to compute the numbers of iterations 18a, 18b, and 18c. The same ratio may be used for all the interatomic distances 14a, 14b, and 14c. A quotient obtained by dividing the time limit 17 by the sum of the estimated execution times 15a, 15b, and 15c may be used as the ratio. Alternatively, different ratios may be used for different interatomic distances. For example, the control unit 12 may use weighted ratios such that the ratio of the limited number of iterations with respect to the estimated number of iterations drops as the interatomic distance increases.

Based on the numbers of iterations 18a, 18b, and 18c, the control unit 12 computes molecular energies 19a, 19b, and 19c corresponding to the interatomic distances 14a, 14b, and 14c, by executing the above-described algorithm. For example, the control unit 12 allows the iteration process of the algorithm until the corresponding one of the numbers of iterations 18a, 18b, and 18c is reached. If the number of iterations 18a, 18b, or 18c is reached, even if the molecular energy has not yet converged, the control unit 12 forcibly stops the corresponding iteration process. In this case, the control unit 12 may output a molecular energy, based on the result of the iteration process executed until the iteration process is stopped. The control unit 12 may output the molecular energy computed by the last iteration.

The control unit 12 outputs the molecular energies 19a, 19b, and 19c in association with the interatomic distances 14a, 14b, and 14c. The control unit 12 may store the molecular energies 19a, 19b, and 19c in a non-volatile storage, may display the molecular energies 19a, 19b, and 19c on a display device, or may transmit the molecular energies 19a, 19b, and 19c to a different information processing apparatus. The control unit 12 may output the above-described other molecular energies corresponding to the above-described other interatomic distances computed by using the different algorithm, along with the molecular energies 19a, 19b, and 19c. The information in which the plurality of interatomic distances and the plurality of molecular energies are associated with each other may be represented in the form of a potential energy curve.

As described above, the information processing apparatus 10 according to the first embodiment computes, based on the molecular information 13, for each of the plurality of interatomic distances, an estimated execution time and an estimated number of iterations of the algorithm that computes a molecular energy by using quantum circuit data. The information processing apparatus 10 determines a limited number of iterations for each of the interatomic distances, based on the time limit 17 and based on the estimated execution time and the estimated number of iterations for each of the plurality of interatomic distances. The information processing apparatus 10 computes the molecular energy for each of the interatomic distances based on the limited number of iterations determined, by executing the algorithm.

In this way, the information processing apparatus 10 is able to compute an accurate molecular energy in view of the specified time limit 17, and to balance the time limit 17 and the accuracy of the molecular energy. Thus, the processing apparatus 10 is able to efficiently compute the molecular energies corresponding to the plurality of interatomic distances through the quantum chemical computation.

The limited number of iterations determined for at least one of the interatomic distances may be less than the corresponding estimated number of iterations. In this way, the total execution time for the plurality of interatomic distances is reduced. For example, the total execution time is reduced to be equal to or less than the time limit 17. In addition, the molecular energies of many interatomic distances are computed by the algorithm using quantum circuit data.

In addition, the information processing apparatus 10 may determines an upper execution time limit for each of the interatomic distances, based on the total estimated execution time and the time limit. The information processing apparatus 10 may determine the upper limit of the number of iterations for each of the interatomic distances, based on the corresponding upper execution time limit determined. In this way, the individual number of iterations is suitably adjusted such that the corresponding execution time falls within a predetermined range, by using the proportional relationship between the execution time and the number of iterations.

In addition, the ratio of the determined limited number of iterations with respect to the estimated number of iterations may differ among a plurality of interatomic distances. In this way, when the tendency of the improvement of the accuracy of the molecular energy with respect to increase in the number of iterations differs, depending on the interatomic distance, the information processing apparatus 10 is able to reduce deterioration of the accuracy of the molecular energy by using the difference in the tendency. In addition, the ratio may be set to be smaller for a longer interatomic distance. In this way, when the interatomic distance is longer, if it takes more time for the molecular energy to completely converge after the molecular energy comes close to a convergence value, the information processing apparatus 10 is able to reduce the execution time while preventing deterioration in accuracy.

In addition, if the number of iterations executed for an interatomic distance reaches the corresponding limited number of iterations determined in advance, the information processing apparatus 10 may forcibly stop the algorithm, and may output a molecular energy based on a result of the iteration process that has been executed before the stop. In this way, a molecular energy is output with the highest possible accuracy within the specified time limit 17.

In addition, the information processing apparatus 10 may compute the molecular energies of other interatomic distances by executing a different algorithm, and may output information about a potential energy curve including the molecular energies computed by the different algorithm. In this way, the information processing apparatus 10 is able to efficiently generate potential energy curve information by selectively using a plurality of algorithms. For example, for the other interatomic distances (for example, short interatomic distances), if the different algorithm is able to compute sufficiently accurate molecular energies within a short time, the above-described algorithm is executed only on the above-described interatomic distance.

Next, a second embodiment will be described. An information processing apparatus 100 according to the second embodiment generates a potential energy curve indicating the relationship between the distance between two target atoms and the ground-state energy of the target molecule by executing a quantum chemical computation. The information processing apparatus 100 is able to execute a plurality of algorithms. At least one of the algorithms may be executed by a different information processing apparatus. The different information processing apparatus may be a quantum computer.

The information processing apparatus 100 may be a client apparatus or a server apparatus. The information processing apparatus 100 may be installed in a data center or may be included in a cloud system. The cloud system may receive a request for a job relating to a quantum chemical computation via a network, and may transmit a generated potential energy curve as a reply. The information processing apparatus 100 may be referred to as a computer, a molecular simulation apparatus, or a quantum chemical computation apparatus. The information processing apparatus 100 corresponds to the information processing apparatus 10 according to the first embodiment.

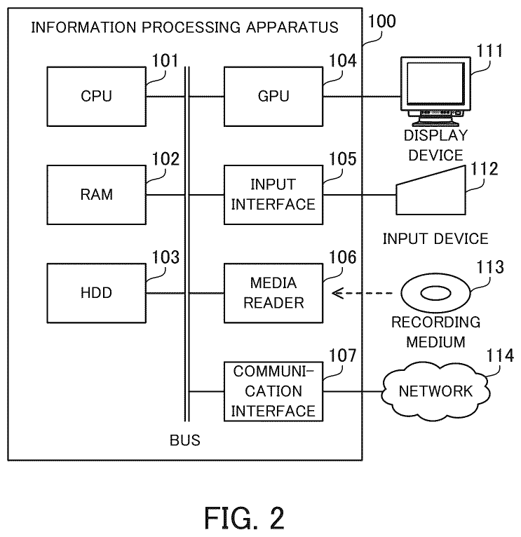

FIG. 2 illustrates a hardware example of the information processing apparatus according to the second embodiment. The information processing apparatus 100 includes a CPU 101, a RAM 102, an HDD 103, a GPU 104, an input interface 105, a media reader 106, and a communication interface 107, all of which are connected to a bus. The CPU 101 corresponds to the control unit 12 according to the first embodiment. The RAM 102 or the HDD 103 corresponds to the storage unit 11 according to the first embodiment.

The CPU 101 is a processor that executes program commands. The CPU 101 loads a program and data stored in the HDD 103 to the RAM 102, and executes the program. The information processing apparatus 100 may include a plurality of processors.

The RAM 102 is a volatile semiconductor memory that temporarily stores a program executed by the CPU 101 and data used for computation by the CPU 101. The information processing apparatus 100 may include a different kind of volatile memory other than a RAM.

The HDD 103 is a non-volatile storage that stores an operating system (OS), middleware, software programs such as application software, and data. The information processing apparatus 100 may include a different kind of non-volatile storage, such as a flash memory or a solid state drive (SSD).

The GPU 104 performs image processing in coordination with the CPU 101, and outputs an image to a display device 111 connected to the information processing apparatus 100. Examples of the display device 111 include a cathode ray tube (CRT) display, a liquid crystal display, an organic electro-luminescence (EL) display, and a projector. A different kind of output device such as a printer may be connected to the information processing apparatus 100.

The GPU 104 may be used as a general-purpose computing on graphics processing unit (GPGPU). The GPU 104 may execute a program in accordance with a command from the CPU 101. The information processing apparatus 100 may include, as a GPU memory, a volatile semiconductor memory other than the RAM 102.

The input interface 105 receives an input signal from an input device 112 connected to the information processing apparatus 100. Examples of the input device 112 include a mouse, a touch panel, and a keyboard. A plurality of input devices may be connected to the information processing apparatus 100.

The media reader 106 is a reading device that reads out a program and data recorded in a recording medium 113. Examples of the recording medium 113 include a magnetic disk, an optical disc, and a semiconductor memory. Examples of the magnetic disk include a flexible disk (FD) and an HDD. Examples of the optical disc include a compact disc (CD) and a digital versatile disc (DVD). The media reader 106 copies a program and data read out from the recording medium 113 to another recording medium such as the RAM 102 or the HDD 103. The read program may be executed by the CPU 101.

The recording medium 113 may be a portable recording medium and may be used for distribution of a program and data. The recording medium 113 and the HDD 103 may each be referred to as a computer-readable recording medium.

The communication interface 107 communicates with other information processing apparatuses via a network 114. The communication interface 107 may be a wired communication interface connected to a wired communication device such as a switch or a router. Alternatively, the communication interface 107 may be a wireless communication interface connected to a wireless communication device such as a base station or an access point.

Next, a quantum chemical computation and a computation algorithm therefor will be described. The quantum chemical computation is a kind of molecular simulation, and analyzes a molecular structure or an intermolecular interaction from an electron state. The quantum chemical computation may be used for assisting material development or drug development. The quantum chemical computation is a microscopic molecular simulation. Although the analysis accuracy of the quantum chemical computation is high, the computation load is also high.

The quantum chemical computation solves a Schradinger equation HΨ=EΨ. H represents the Hamiltonian, W represents the wave function, and E represents the energy. The Hamiltonian H depends on the target molecular structure. The wave function Ψ corresponds to the eigenstate of the electron, and the energy E corresponds to the eigenenergy corresponding to T. The quantum chemical computation computes the ground-state energy obtained when the molecular structure is stable. However, it is difficult to directly solve the Schradinger equation.

Thus, in the quantum chemical computation, the wave function Ψ is expressed by basis functions. The basis functions are a linear combination of known functions. Each of the plurality of terms included in the basis functions corresponds to a molecular orbital. One of the electrons of a molecule is placeable on a molecular orbital. The quantum chemical computation receives molecular information indicating the locations of the plurality of atoms of a molecule, a computation algorithm, and basis functions specified by the user, and computes a ground-state energy based on the specified information. However, in the second embodiment, the computation algorithm may be omitted to be specified. The information processing apparatus 100 generates a potential energy curve based on the quantum chemical computation.

The potential energy curve indicates potential energies corresponding to different interatomic distances. The individual potential energy is the energy of a molecule, assuming that each of the atoms remains stationary. The horizontal axis of the potential energy curve represents the interatomic distance. The vertical axis of the potential energy curve represents the ground-state energy.

The unit of the distance is, for example, angstrom (Å). The unit of the energy is, for example, Hartree. The energy is computed for each of the plurality of discrete distances within a certain range. These distances may be set at equal intervals. For example, the energy is computed at 0.1 Å intervals from 0.8 Å to 2.8 Å. By plotting the computed energies and connecting the computed energies with a line, a potential energy curve is generated. The minimum point of the potential energy curve may represent the most stable state of the molecule. The maximum point of the potential energy curve may represent a transition state of the molecule.

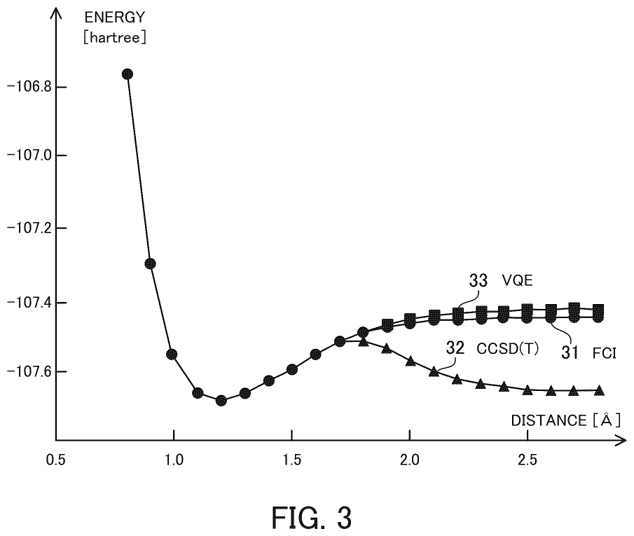

FIG. 3 illustrates accuracy examples of potential energy curves. The second embodiment assumes that CCSD(T) and VQE are selectively used as the quantum chemical computation algorithms. CISD or CCSD may alternatively be used for CCSD(T). In addition, FIG. 3 illustrates full configuration interaction (FCI) as a very accurate algorithm.

A curve 31 is a potential energy curve generated by FCI alone. A curve 32 is a potential energy curve generated by CCSD(T) alone. A curve 33 is a potential energy curve generated by VQE alone.

FCI is a classical algorithm assumed to be executed by a classical computer. FCI computes an exact solution of an energy based on specified molecular information and basis functions. Thus, although the accuracy of the solution obtained by FCI is high, the execution time is long. FCI needs a computation amount that is on the order of the factorial of the number of molecular orbitals. Thus, it is difficult for FCI to compute the energy of a large-scale molecule. Because FCI computes an exact solution, there are cases in which an energy computed by FCI is interpreted as a correct energy.

CCSD(T) is a classical algorithm assumed to be executed by a classical computer. CCSD(T) computes an approximate solution of an energy based on specified molecular information and basis functions. Thus, although the accuracy of the solution obtained by CCSD(T) is lower than that of FCI, the execution time of CCSD(T) is shorter than that of FCI. CCSD(T) needs a computation amount on the order of the seventh power of the number of molecular orbitals. The accuracy of the solution obtained by CCSD is lower than that of CCSD(T), and the execution time of CCSD is shorter.

CCSD(T) executes an exact computation of the impact of a single-electron excitation and a two-electron excitation as an electron state on the energy, and computes the impact of a three-electron excitation on the energy from perturbation. CCSD(T) ignores the impact of high-order excitations such as a four-electron excitation or greater. CCSD(T) iteratively computes the energy while changing the electron configuration, and searches for a minimum energy. CCSD(T) executes the iterative computation until the computed energy converges. For example, CCSD(T) compares the latest energy with the energy computed by the previous iteration. If the difference between these energies is less than a threshold, CCSD(T) stops the iteration process.

In many cases, CCSD(T) computes relatively good approximate solutions for shorter interatomic distances, even when compared with those of FCI. However, for longer interatomic distances, there are cases in which CCSD(T) computes inaccurate approximate solutions. This is because, when the interatomic distance is longer, outer molecular orbitals affect the energy more greatly. That is, the approximate solutions of CCSD(T), which ignores the impact of high-order electron excitations such as a four-electron excitation or greater, have larger errors for longer interatomic distances. In addition, in the case of CCSD(T), when the accuracy of the finally outputted energy is low, a greater number of iterations tends to be executed until the energy converges. This is because even when the iterative computation is executed, the approximate solution could continue to vary around a correct value, and the approximate solution does not stably converge to the correct value.

VQE is a quantum algorithm assumed to be executed by a gate-based quantum computer. However, VQE is executable on a classical computer by using a quantum simulator. The quantum simulator simulates an operation of a quantum computer by using software. In this case, when the qubit increases by one bit, the memory usage and the computation amount of the classical computer are doubled. The second embodiment assumes that VQE is executed by using a quantum simulator. The accuracy of the solution of VQE and the execution time of VQE are between FCI and CCSD(T). That is, the accuracy of the solution obtained by VQE is less than FCI and is greater than CCSD(T). The execution time is shorter than FCI and longer than CCSD(T).

VQE creates a quantum circuit that generates a quantum state by using a plurality of qubits, based on specified basis functions. This quantum circuit may be referred to as an ansatz circuit. In addition, VQE creates a quantum circuit that measures an energy from the quantum state, based on the Hamiltonian corresponding to specified molecular information. This quantum circuit may be referred to as a measurement circuit. These quantum circuits are each a quantum computation model written by a combination of quantum gates. In a quantum computer, a quantum circuit is implemented by using physical qubits. In a quantum simulator, pseudo qubit data is stored in a memory, and pseudo quantum gate operations are implemented by using a classical program.

VQE generates a quantum state by using the ansatz circuit, and measures an energy by using the measurement circuit. The individual measured values are affected by noise and fluctuation. For the same electron configuration, VQE executes generation of a quantum state and measurement of an energy a plurality of times, and computes an average value of the energies as an energy expectation value. VQE varies the parameter values for generating a quantum state such that the energy expectation value drops. Varying the parameter values corresponds to varying the electron configuration. VQE iterates the above process, so as to search for the ground-state energy. For example, VQE iterates the above-described process until the energy expectation value converges.

The “classical computer” is, for example, a von Neumann computer as opposed to “quantum computer”. The “classical algorithm” is, for example, an algorithm as opposed to “quantum algorithm”, and does not use a quantum circuit.

As indicated by the curves 32 and 33, for long interatomic distances, there are cases in which the accuracy of CCSD(T) is significantly lower than that of VQE. However, the execution time of VQE is significantly longer than that of CCSD(T). For example, there are cases in which the execution time of VQE exceeds 1000 times of that of CCSD(T). In this respect, the information processing apparatus 100 is incapable of ignoring the execution time needed for generating a potential energy curve, and the user may request to generate a potential energy curve within a specified time limit.

Thus, the information processing apparatus 100 automatically selects an algorithm and generates a potential energy curve as accurate as possible within a time limit specified by the user. The information processing apparatus 100 adopts the energies obtained by CCSD(T) for short distances, and adopts the energies obtained by VQE for long distances. In this case, the distance at which the accuracy of CCSD(T) beings to drop significantly varies depending on the kind of the molecule. Thus, the information processing apparatus 100 automatically selects the VQE target distances from the execution result of CCSD(T).

In the second embodiment, first, the information processing apparatus 100 executes CCSD(T) for all the distances. The second embodiment assumes that the execution time of CCSD(T) is negligibly short. Next, the information processing apparatus 100 analyzes the number of iterations of CCSD(T) for each distance, detects a boundary point at which the accuracy of CCSD(T) begins to drop, and selects the distance at the boundary point and the distances thereafter as the VQE targets. The information processing apparatus 100 additionally executes VQE on the selected distances.

In this case, for each of the selected distances, the information processing apparatus 100 estimates the execution time and the number of iterations normally needed by VQE until the energy converges. When VQE is normally executed on all the selected distances, if the total execution time exceeds the time limit, the information processing apparatus 100 limits the number of VQE iterations such that the time limit is not exceeded. If the number of VQE iterations is limited, there are cases in which VQE is stopped before the energy converges.

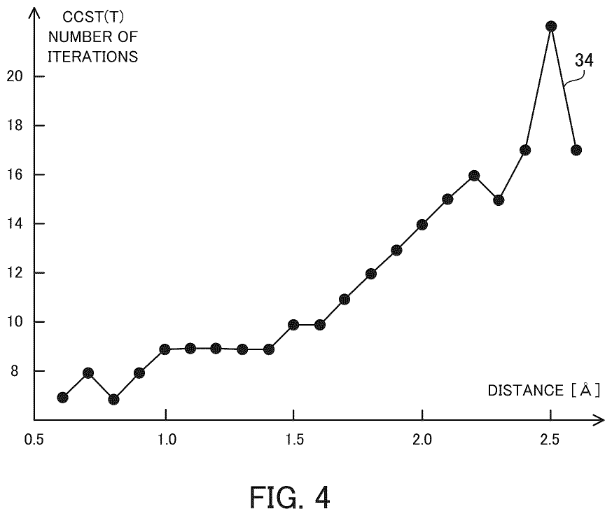

Next, detection of a boundary point will be described. FIG. 4 illustrates an example of the relationship between the interatomic distance and the number of iterations of a classical algorithm. A curve 34 illustrates a relationship between the interatomic distance and the number of iterations of CCSD(T). As described above, when the distance is longer, CCSD(T) indicates a lower accuracy and needs a greater number of iterations. When the accuracy of CCSD(T) beings to drop, the number of iterations of CCSD(T) significantly rises.

Thus, when CCSD(T) outputs the energies corresponding to the individual distances, the information processing apparatus 100 measures and records the numbers of iterations of CCSD(T) for each distance. The information processing apparatus 100 scans the number of iterations of CCSD(T) in the ascending order of the distances. When the information processing apparatus 100 detects a significant rise in the number of iterations, the information processing apparatus 100 determines that the accuracy of CCSD(T) has begun to drop.

For example, based on a certain number of recent distances and the numbers of iterations corresponding thereto (for example, five recent distances and the numbers of iterations corresponding thereto), the information processing apparatus 100 executes fitting to establish a regression line based on a least squares method, and computes the slope of the regression line. The information processing apparatus 100 determines whether the most recent slope is greater than the slope computed at the previous distance. If the slope consecutively rises a certain number of times (for example, three consecutive times), the information processing apparatus 100 determines the distance at this point of time to be the boundary point. The information processing apparatus 100 selects the distance at the boundary point and the longer distances thereafter as the VQE targets. However, the distance at the boundary point may be excluded from the VQE targets. The information processing apparatus 100 may select the distances greater than the distance at the boundary point as the VQE targets.

In the example of the curve 34, at a distance of 1.4 Å, a regression line having a slope of 0 is computed from the numbers of iterations corresponding to the distances of 1.0 Å to 1.4 Å. At a distance of 1.5 Å, a regression line having a positive slope is computed from the numbers of iterations corresponding to the distances of 1.1 Å to 1.5 Å. At a distance of 1.6 Å, a regression line having a slope greater than that obtained at the distance of 1.5 Å is computed from the numbers of iterations corresponding to the distances of 1.2 Å to 1.6 Å. At a distance of 1.7 Å, a regression line having a slope greater than that obtained at the distance of 1.6 Å is computed from the numbers of iterations corresponding to the distances of 1.3 Å to 1.7 Å. Thus, the information processing apparatus 100 selects the distance of 1.7 Å (or 1.8 Å) and the distances thereafter as the VQE targets.

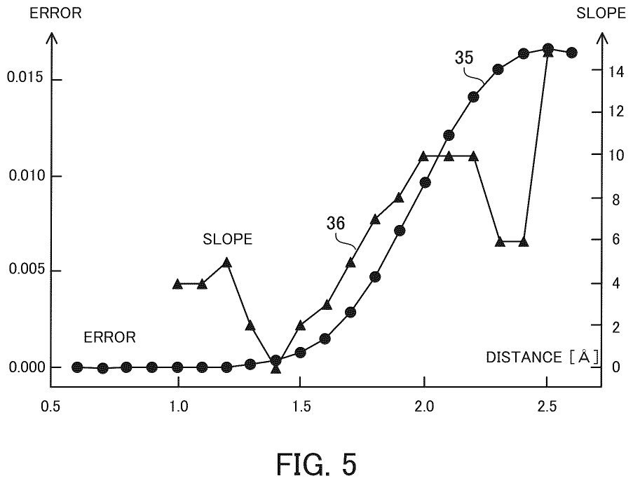

FIG. 5 illustrates an example of the error of the classical algorithm and an example of the slope of the number of iterations. A curve 35 indicates the absolute value of the error of the energy computed by CCSD(T) with respect to the energy computed by FCI. As indicated by the curve 35, the error of CCSD(T) sharply rises from the distance of 1.5 Å to the distance of 2.0 Å. A curve 36 indicates the slope of the numbers of iterations at the five recent points. As indicated by the curve 36, the slope of the numbers of iterations of CCSD(T) consecutively rises from the distance of 1.5 Å to the distance of 2.0 Å. Thus, the information processing apparatus 100 is able to detect whether the error has begun to rise significantly, by monitoring the slope of the numbers of iterations.

Next, estimation of an individual VQE execution time will be described. The information processing apparatus 100 estimates the individual VQE execution time by using a machine learning model trained in advance. The machine learning model may be referred to as an estimator. The machine learning model according to the second embodiment is a Gaussian process regression model generated by a Gaussian process. The machine learning for training this machine learning model may be executed by the information processing apparatus 100 or another information processing apparatus.

The machine learning model includes a time model that estimates the execution time per iteration of VQE and an iteration model that estimates the number of iterations of VQE. The execution time per iteration corresponds to the time needed for computing the energy expectation value corresponding to a single electron configuration. The number of iterations corresponds to the number of trials needed for changing the electron configuration. An estimated value of the VQE execution time is a product of the execution time estimated by the time model and the number of iterations estimated by the iteration model.

However, the actual number of iterations may fluctuate due to randomness, and there is a risk that the number of iterations may exceed an expectation value. In addition, due to a small volume of training data, uncertainty could occur in the result estimated by the iteration model. Thus, the information processing apparatus 100 may use an iteration model that outputs the number of iterations greater than an expectation value in view of at least one of the randomness and uncertainty. Hereinafter, example machine learning models will be described with expressions.

First, a time model that estimates the execution time per iteration will be described. The explanatory variable of the time model is a vector x of degree 3, as expressed by Expression (1). In Expression (1), q represents the number of qubits, d represents the depth of the ansatz circuit, and 1 represents the number of terms of the Hamiltonian. The depth of the ansatz circuit is the number of stages of quantum gates arranged in series. The number of terms of the Hamiltonian is the number of terms obtained when the Hamiltonian is broken down to a sum of Pauli matrices.

$$x = ( q , d , ℓ ) \qquad ( 1 )$$

A time model that computes an expectation value of the execution time per iteration is defined as expressed by Expression (2), for example. In Expression (2), y represents a response variable indicating the execution time per iteration, and n represents the number of records included in the training data. The training data for training the time model includes n records, each of which is a pair of an explanatory variable and a response variable, such as (x₁, y₁), . . . , (xₙ, yₙ).

$$y = ( {y}_{1} , … , {y}_{n} ) {( {K}_{n} + λ {I}_{n} )}^{- 1} {k}_{n} ( x ) \qquad ( 2 )$$

k is the kernel of the Gaussian process. The kernel k is a function that defines the similarity between vectors. Examples of the kernel k include a radial basis function (RBF) kernel and a Matern kernel. Kₙ in Expression (2) is an n×n square matrix generated from explanatory variables included in the training data. The component of the i-th row and the j-th column of the matrix Kₙ is k(xᵢ, xⱼ). The matrix Kₙ indicates the similarity between two explanatory variables included in the training data. Iₙ represents an n×n identity matrix. kₙ(x) is a column vector of which the i-th row component is k(xᵢ, x). kₙ (x) indicates the similarity of a certain vector x to each of the n explanatory variables included in the training data. λ is a constant greater than 0.

The information processing apparatus 100 may take into account the risk that the actual execution time per iteration fluctuates from an expectation value, and may use a time model that takes robustness against the risk into account. First, as expressed by Expression (3), a conditional value at risk (CVaR) is defined for the execution time per iteration. In Expression (3), α is a constant that is greater than 0 and is equal to or less than 1. ψᵥ(y) and U are defined as expressed by Expression (4).

$${CVaR}_{n , α} ( x ; {y}_{1} , … , {y}_{n} ) = {max}_{v ∈ U} \{ v - \frac{1}{α} ( {ψ}_{v} ( {y}_{1} ) , … , {ψ}_{v} ( {y}_{n} ) ) {( {K}_{n} + λ {I}_{n} )}^{- 1} {k}_{n} ( x ) \} \qquad ( 3 )$$

$${ψ}_{v} ( y ) = max ( v - y , 0 ) , U = \{ {y}_{1} , … , {y}_{n} \} \qquad ( 4 )$$

The time model that takes the robustness into account is defined as expressed by Expression (5) by using CvaR of Expression (3), for example. The risk that the execution time per iteration rises is reflected on an estimated value computed by Expression (5), and is assumed to be greater than the expectation value computed by Expression (2). Assuming that the distribution about the vector x is represented by ρ and a cumulative distribution function corresponding to the distribution ρ is represented by F, Expression (5) gives an estimated value of Expression (6).

$$- {CVaR}_{n , 1 - α} ( x ; - {y}_{1} , … , - {y}_{n} ) \qquad ( 5 )$$

$${E}_{y ∼ ρ ( x )} [ y \vert y \geq {F}^{- 1} ( α ) ] \qquad ( 6 )$$

In addition, the information processing apparatus 100 may further take into account uncertainty of the estimation of the time model due to insufficient training data, and may use a time model that takes the robustness and uncertainty into account. First, as expressed by Expression (7), about the execution time pet iteration, σₙ(x) is defined. In Expression (7), k^{T}ₙ(x) represents a transpose matrix of kₙ(x).

$${σ}_{n} ( x ) = \sqrt{k ( x , x ) - {k}_{n}^{T} ( x ) {K}_{n}^{- 1} {k}_{n} ( x )} \qquad ( 7 )$$

The time model that takes the robustness and uncertainty into account is defined as expressed by Expression (8) by using σₙ(x) of Expression (7), for example. In Expression (8), β is a positive constant. The risk that the execution time per iteration further rises is reflected on an estimated value computed by Expression (8), and the estimated value is greater than the estimated value computed by Expression (5).

$$- {CVaR}_{n , 1 - α} ( x ; - {y}_{1} , … , - {y}_{n} ) + {βσ}_{n} ( x ) \qquad ( 8 )$$

Next, an iteration model that estimates the number of iterations will be described. The basic configuration of the iteration model is the same as that of the time model. However, the meanings of the explanatory variable and response variable differ from those of the time model. The explanatory variable of the iteration model is a vector z of degree 2, as expressed by Expression (9). In Expression (9), m represents the number of iterations of the classical algorithm, and s represents the interatomic distance.

In the second embodiment, the classical algorithm is CCSD(T). Alternatively, the classical algorithm may be CISD or CCSD. It may be interpreted that “CCSD” in the broad sense includes CCSD in the narrow sense and CCSD(T).

$$z = ( m , s ) \qquad ( 9 )$$

The iteration model that estimates the number of iterations is defined as expressed by Expression (10), for example. In Expression (10), w represents a response variable indicating the number of iterations of VQE. The training data for training the iteration model includes n records, each of which is a pair of an explanatory variable and a response variable, such as (z₁, w₁), . . . , (zₙ, wₙ).

$$w = ( {w}_{1} , … , {w}_{n} ) {( {L}_{n} + λ {I}_{n} )}^{- 1} {ℓ}_{n} ( z ) \qquad ( 10 )$$

In Expression (10), 1 represents the kernel of the Gaussian process. Lₙ represents an n×n square matrix generated from explanatory variables included in the training data. The component in the i-th row and the j-th column of the matrix Lₙ is expressed by l(zᵢ, zⱼ). lₙ(z) represents a column vector in which the component of the i-th row is expressed by l(zᵢ, z). A represents a constant greater than 0.

As is the case with the time model, the information processing apparatus 100 may take into account the risk that the actual number of iterations fluctuates from the expectation value, and may use an iteration model that takes robustness against this risk into account. The iteration model that takes the robustness into account is defined as expressed by Expression (11) by using CVaR in Expression (3), for example. However, regarding Expression (3) and Expression (4), x is replaced by z, y is replaced by w, Kₙ is replaced by Lₙ, and kₙ is replaced by lₙ.

$$- {CVaR}_{n , 1 - α} ( z ; - {w}_{1} , … , - {w}_{n} ) \qquad ( 11 )$$

In addition, the information processing apparatus 100 may further take into account uncertainty of the estimation of the iteration model due to insufficient training data and may use an iteration model that takes the robustness and uncertainty into account. The iteration model that takes the robustness and uncertainty into account is defined as expressed by Expression (12) by using Expression (7), for example. However, regarding Expression (7), x is replaced by z, Kₙ is replaced by Lₙ, and kₙ is replaced by lₙ.

$$- {CVaR}_{n , 1 - α} ( z ; - {w}_{1} , … , - {w}_{n} ) + {βσ}_{n} ( z ) \qquad ( 12 )$$

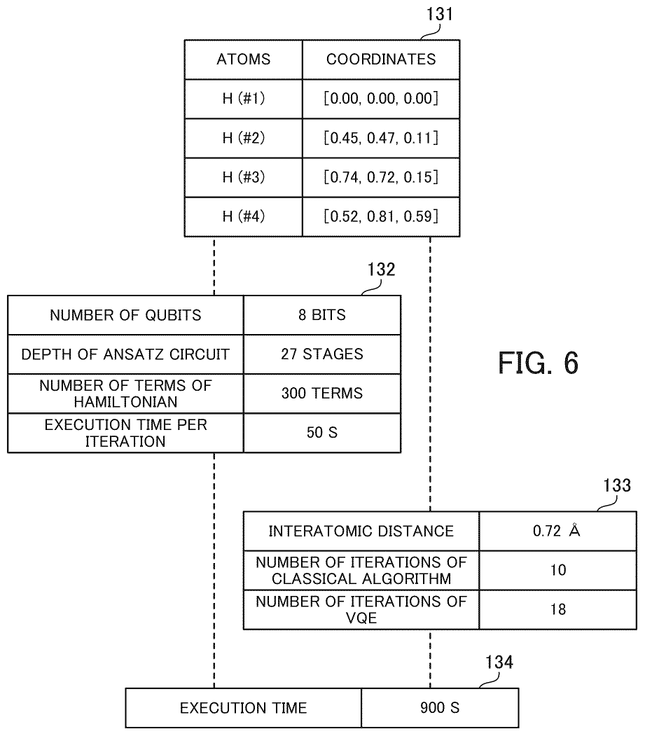

FIG. 6 illustrates an example of estimation of an execution time of VQE. Hereinafter, a process for computing the energy corresponding to a single distance based on VQE may be referred to as a VQE job. The information processing apparatus 100 acquires data 131 about an analysis target molecule. The data 131 indicates the kind and coordinates of each of the plurality of atoms of the molecule. When machine learning is executed, n data sets, each of which corresponds to the data 131, are used as sample data.

The information processing apparatus 100 generates data 132 from the data 131. The data 132 includes the number of qubits, the depth of the ansatz circuit, the number of terms of the Hamiltonian, and the execution time per iteration. The number of qubits, the depth of the ansatz circuit, and the number of terms of the Hamiltonian are input data of the time model, and are computed from the data 131 in the preprocessing of VQE. The execution time per iteration is output data of the time model.

When machine learning is executed, n data sets, each of which corresponds to the data 132, are used as the training data for training the time model. In this case, the execution time per iteration corresponds to a correct label, and is measured by executing VQE on the molecular information about the samples.

In addition, the information processing apparatus 100 generates data 133 from the data 131. The data 133 includes an interatomic distance, the number of iterations of the classical algorithm, and the number of iterations of VQE. The interatomic distance and the number of iterations of the classical algorithm are input data of the iteration model. The number of iterations of the classical algorithm is measured by executing a classical algorithm based on the data 131. In the second embodiment, the number of iterations of the classical algorithm is, for example, the number of iterations of CCSD(T). The number of iterations of VQE is output data of the iteration model.

When machine learning is executed, n data sets, each of which is corresponds to the data 133, are used as the training data for training the iteration model. In this case, the number of iterations of VQE corresponds to a correct label, and is measured by executing VQE.

The information processing apparatus 100 generates data 134 from the data 132 and 133. The data 134 includes an estimated VQE execution time. The execution time is a product of the execution time per iteration included in the data 132 and the number of VQE iterations included in the data 133. One or both of the execution time per iteration that is output by the time model and the number of VQE iterations that is output by the iteration model may be an expectation value, an estimated value obtained in view of the robustness, or an estimated value obtained in view of the robustness and uncertainty. The information processing apparatus 100 may switch the kind of the estimated value, based on a command from the user.

In this way, the information processing apparatus 100 computes an estimated VQE execution time and an estimated number of VQE iterations before execution of VQE, for each of the VQE target distances. Next, adjustment of the individual VQE execution times will be described. The information processing apparatus 100 sets an upper limit of the number of VQE iterations for each distance such that the energies for all the VQE target distances are computed within a time limit, and adjusts the VQE execution time for each distance, based on the upper limit.

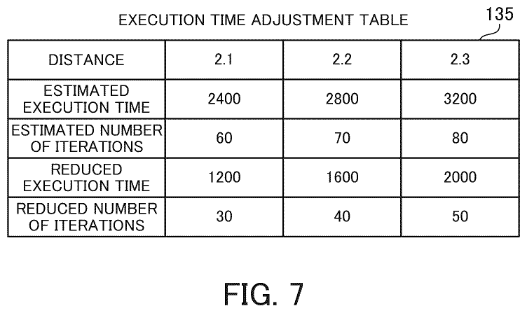

FIG. 7 illustrates a first example of adjustment of VQE execution times. An execution time adjustment table 135 indicates reduction of the execution time and reduction of the number of iterations for each distance. The reduction of the execution time may be referred to as contraction of the execution time, and the reduction of the number of iterations may be referred to as contraction of the number of iterations. For ease of description, this example assumes that three distances, which are a distance of 2.1 Å, a distance of 2.2 Å, and a distance of 2.3 Å, are selected as the VQE target distances.

The estimated execution time at the distance of 2.1 Å is 2400 seconds, and the estimated number of iterations at the distance of 2.1 Å is 60 times. The estimated execution time at the distance of 2.2 Å is 2800 seconds, and the estimated number of iterations at the distance of 2.2 Å is 70 times. The estimated execution time at the distance of 2.3 Å is 3200 seconds, and the estimated number of iterations at the distance of 2.3 Å is 80 times. Thus, the estimated execution time per iteration is 40 seconds, which is common to the distance of 2.1 Å, the distance of 2.2 Å, and the distance of 2.3 Å. However, there are cases in which the estimated execution time per iteration differs depending on the distance.

The present example assumes that the user sets 4800 seconds as a specified time limit. The total estimated execution time is 8400 seconds, and if the VQE is normally executed, VQE will probably not end within the time limit. Thus, the information processing apparatus 100 refers to the estimated execution time with respect to each distance, and determines a reduced execution time for each distance. Based on the ratio of the individual reduced execution time with respect to the corresponding estimated execution time, the information processing apparatus 100 determines a reduced number of iterations from the corresponding estimated number of iterations. The individual reduced number of iterations is set as the upper limit of the corresponding number of VQE iterations.

For example, the information processing apparatus 100 determines the reduced execution time for the distance of 2.1 Å to be 1200 seconds, the reduced execution time for the distance of 2.2 Å to be 1600 seconds, and the reduced execution time for the distance of 2.3 Å to be 2000 seconds. The sum of the reduced execution times is 4800 seconds, which is within the time limit. For the distance of 2.1 Å, the information processing apparatus 100 multiplies the estimated number of iterations by the ratio of the reduced execution time with respect to the estimated execution time, the ratio being 50%, and determines the reduced number of iterations to be 30.

In addition, for the distance of 2.2 Å, the information processing apparatus 100 multiplies the estimated number of iterations by the ratio of the reduced execution time with respect to the estimated execution time, the ratio being 57%, and determines the reduced number of iterations to be 40. In addition, for the distance of 2.3 Å, the information processing apparatus 100 multiplies the estimated number of iterations by the ratio of the reduced execution time with respect to the estimated execution time, the ratio being 63%, and determines the reduced number of iterations to be 50. Alternatively, the information processing apparatus 100 may compute the reduced number of iterations by dividing the corresponding reduced execution time by the estimated execution time per iteration.

Next, distribution of the reduced execution times among the plurality of VQE target distances will be described. As an example of the distribution method, there is uniform distribution in which the same ratio of the reduced execution time with respect to the estimated execution time is used for all the VQE target distances. As another example of the distribution method, there is weighted distribution in which the ratio of the reduced execution time with respect to the estimated execution time is varied depending on the VQE target distance.

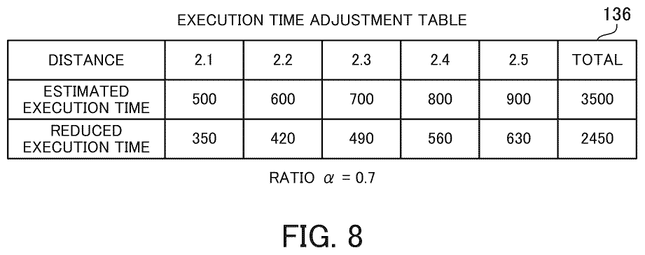

FIG. 8 illustrates a second example of adjustment of VQE execution times. An execution time adjustment table 136 indicates reduction of the execution time for each distance. In FIG. 8, five distances, which are from a distance of 2.1 Å to a distance of 2.5 Å, are selected as the VQE target distances.

The estimated execution time at a distance of 2.1 Å is 500 seconds, the estimated execution time at a distance of 2.2 Å is 600 seconds, the estimated execution time at a distance of 2.3 Å is 700 seconds, the estimated execution time at a distance of 2.4 Å is 800 seconds, and the estimated execution time at a distance of 2.5 Å is 900 seconds. Thus, the total estimated execution time is 3500 seconds. The present example assumes that the user sets 2500 seconds as the specified time limit. The information processing apparatus 100 determines the reduced execution time for each distance based on the uniform distribution.

In this case, the information processing apparatus 100 computes a ratio α that is common to all the VQE target distances as 0.7, by dividing the time limit by the total estimated execution time. The information processing apparatus 100 determines the individual reduced execution time for each distance by multiplying the corresponding estimated execution time by the ratio α. Thus, the reduced execution time at the distance of 2.1 Å is 350 seconds, the reduced execution time at the distance of 2.2 Å is 420 seconds, the reduced execution time at the distance of 2.3 Å is 490 seconds, the reduced execution time at the distance of 2.4 Å is 560 seconds, and the reduced execution time at the distance of 2.5 Å is 630 seconds. Thus, the total reduced execution time is 2450 seconds and falls within 2500 seconds, which is the time limit specified by the user.

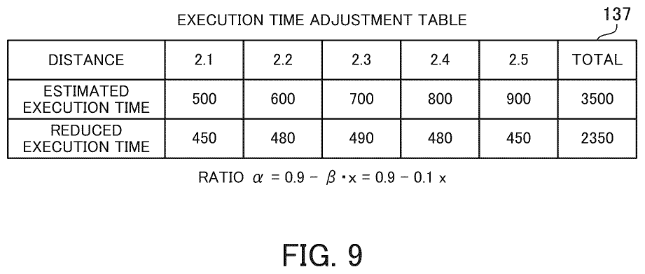

FIG. 9 illustrates a third example of adjustment of VQE execution times. An execution time adjustment table 137 indicates reduction of the execution time for each distance. The VQE target distances, the estimated execution times, and the time limit are the same as those in the execution time adjustment table 136. However, the information processing apparatus 100 determines the reduced execution times based on the weighted distribution, not the uniform distribution.

In VQE, when the interatomic distance is longer, after the energy comes close to a convergence value, the waiting time needed until the energy completely converges and the algorithm stops tends to be longer. Thus, when the interatomic distance is longer, even if the ratio α of the reduced execution time with respect to the estimated execution time is set to be small, the accuracy of the computed energy is little affected. Therefore, the information processing apparatus 100 determines the reduced execution times by setting a smaller ratio α for a longer VQE target distance.

For example, the information processing apparatus 100 computes the ratio α for each VQE target distance based on a linear form expressed by 0.9−β×x. The ratio α may be computed based on a nonlinear form. x is a non-negative integer index indicating the order of the plurality of VQE target distances. The shortest VQE target distance is indicated by x=0, the second shortest VQE target distance is indicated by x=1. Thereafter, as the VQE target distance is extended by one level, x is incremented by 1. β is a non-negative coefficient determined from the relationship between the estimated execution time and the time limit.

For example, the information processing apparatus 100 begins with β=0 and gradually increases β while comparing the total reduced execution time with the time limit. As β rises, the total reduced execution time drops. The information processing apparatus 100 searches for a minimum β that drops the total reduced execution time to the time limit or less, and determines the reduced execution time for each VQE target distance by using β. For example, the information processing apparatus 100 determines that β=0.1.

In FIG. 9, the ratio α at the distance of 2.1 Å is 0.9, and the reduced execution time is 450 seconds. The ratio α at the distance of 2.2 Å is 0.8, and the reduced execution time is 480 seconds. The ratio α at the distance of 2.3 Å is 0.7, and the reduced execution time is 490 seconds. The ratio α at the distance of 2.4 Å is 0.6, and the reduced execution time is 480 seconds. The ratio α at the distance of 2.5 Å is 0.5, and the reduced execution time is 450 seconds. The total reduced execution time is 2350 seconds and falls within 2500 seconds, which is the time limit specified by the user.

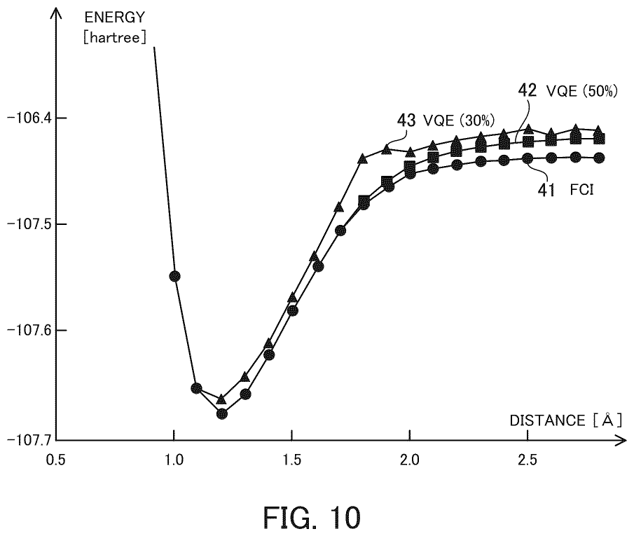

FIG. 10 illustrates accuracy examples of potential energy curves when reduction in execution time is executed. A curve 41 is a potential energy curve indicating the energy computed by FCI, and is interpreted as a correct potential energy curve.

A curve 42 is a potential energy curve indicating the energy computed by VQE in which the total execution time is limited to 50% of the normal operation. Because a potential energy curve that indicates the energy computed by VQE in which the total execution time is not limited approximates the curve 42, this curve is not illustrated in FIG. 10. A curve 43 is a potential energy curve indicating the energy computed by VQE in which the total execution time is limited to 30% of the normal operation.

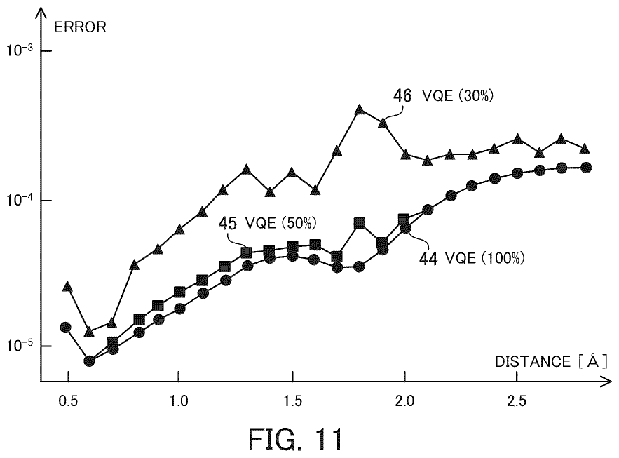

FIG. 11 illustrates examples of VQE errors. A curve 44 indicates the error of the energy computed by VQE in which the execution time is not limited, with respect to the energy computed by FCI. A curve 45 indicates the error of the energy computed by VQE in which the total execution time is limited to 50% of the normal operation, with respect to the energy computed by FCI. A curve 46 indicates the error of the energy computed by VQE in which the total execution time is limited to 30% of the normal operation, with respect to the energy computed by FCI.

As indicated by the curves 41 to 46, even if the interatomic distance is long, it is possible to compute an accurate energy by using VQE. In addition, even if the total execution time of VQE is reduced by approximately 50%, the accuracy of the computed energy is little affected. Next, functions of the information processing apparatus 100, and the procedure of a quantum chemical computation executed by the information processing apparatus 100 will be described.

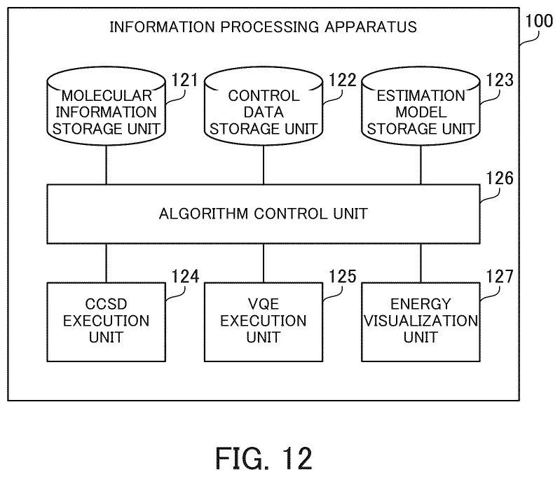

FIG. 12 is a block diagram illustrating an example of functions of the information processing apparatus. The information processing apparatus 100 includes a molecular information storage unit 121, a control data storage unit 122, and an estimation model storage unit 123. These storage units are each implemented by using the RAM 102 or the HDD 103, for example.

The information processing apparatus 100 also includes a CCSD execution unit 124, a VQE execution unit 125, an algorithm control unit 126, and an energy visualization unit 127. These processing units are implemented by using the CPU 101 and a program, for example. One or both of the CCSD execution unit 124 and the VQE execution unit 125 may be included in another information processing apparatus.

The molecular information storage unit 121 stores molecular information. The molecular information includes the kinds and positional coordinates of the atoms of a simulation target molecule. The positional coordinates of the individual atoms are corrected based on the distance between two target atoms. In addition, the molecular information storage unit 121 stores basis functions specified by the user. In many cases, the basis functions are selected by the user from a group of known basis functions based on the kind of the molecule or the purpose of the molecular simulation.

The control data storage unit 122 stores a plurality of distances for which their respective energies are computed. In addition, for each distance, the control data storage unit 122 stores the energy computed by the classical algorithm and the number of iterations of the classical algorithm. In addition, the control data storage unit 122 stores, for each VQE target distance, the estimated VQE execution time and the estimated number of VQE iterations. In addition, the control data storage unit 122 stores the energy computed by the VQE for each VQE target distance.

The estimation model storage unit 123 stores a time model that estimates the execution time per iteration from features of a quantum circuit. In addition, the estimation model storage unit 123 stores an iteration model that estimates the number of iterations of VQE from the interatomic distance and the number of iterations of the classical algorithm. The time model and the iteration model are trained by the information processing apparatus 100 or another information processing apparatus.

Upon receiving a command from the algorithm control unit 126, the CCSD execution unit 124 executes the classical algorithm based on molecular information and basis functions specified. For example, CCSD(T) is executed as the classical algorithm. CCSD may be executed alternatively. The CCSD execution unit 124 computes a ground-state energy for each item of molecular information corresponding to a single distance and outputs the ground-state energy to the algorithm control unit 126. In addition, the CCSD execution unit 124 measures the number of iterations and notifies the algorithm control unit 126 of the measurement result.

Upon receiving a command from the algorithm control unit 126, the VQE execution unit 125 executes VQE based on molecular information and basis functions specified. Based on the molecular information and basis functions, the VQE execution unit 125 generates a quantum circuit as preprocessing, and outputs the generated quantum circuit to the algorithm control unit 126. In addition, the VQE execution unit 125 iterates the measurement of the energy by using the quantum circuit, computes a ground-state energy for each item of molecular information corresponding to a single VQE target distance, and outputs the ground-state energy to the algorithm control unit 126.

The upper limit of the number of iterations may be specified from the algorithm control unit 126. In this case, the VQE execution unit 125 executes VQE such that the number of iterations does not exceed the upper limit. If the energy does not satisfy a convergence condition even after the number of iterations reaches the upper limit, the VQE execution unit 125 forcibly stops the VQE and outputs the energy obtained last which has not yet converged.

The algorithm control unit 126 receives a time limit specified by the user. The algorithm control unit 126 determines VQE target distances based on the execution result of the classical algorithm, and limits the number of iterations for each VQE target distance such that the total execution time is equal to or less than the time limit.

First, the algorithm control unit 126 causes the CCSD execution unit 124 to compute the energies for all the distances, and acquires the individual energies and numbers of iterations based on the classical algorithm. The algorithm control unit 126 scans the numbers of iterations of the classical algorithm in the ascending order of the distances, detects a boundary point at which the number of iterations sharply rises, and selects the distance at the boundary point and the distances thereafter as the VQE target distances.

Next, for each VQE target distance, the algorithm control unit 126 causes the VQE execution unit 125 to execute preprocessing and acquires features of the structure of the quantum circuit. The algorithm control unit 126 estimates the execution time and the number of iterations for each VQE target distance, from the features of the quantum circuit and the numbers of iterations of the classical algorithm, by using a machine learning model stored in the estimation model storage unit 123.

The algorithm control unit 126 determines a reduced number of iterations for each VQE target distance in accordance with a certain computation method, from the corresponding estimated execution time and the corresponding estimated number of iterations for each VQE target distance and the specified time limit. The algorithm control unit 126 causes the VQE execution unit 125 to execute VQE such that the number of iterations does not exceed the corresponding reduced number of iterations.

The energy visualization unit 127 generates a potential energy curve by retrieving a plurality of energies corresponding to a plurality of distances from the control data storage unit 122 and by plotting the retrieved energies. The energy visualization unit 127 uses the energies of VQE for the distances on which the VQE has been executed, and uses the energies of the classical algorithm for the distances on which VQE has not been executed.

The energy visualization unit 127 outputs a generated potential energy curve. The energy visualization unit 127 may store the potential energy curve in a non-volatile storage, may display the potential energy curve on the display device 111, or may transmit the potential energy curve to another information processing apparatus.

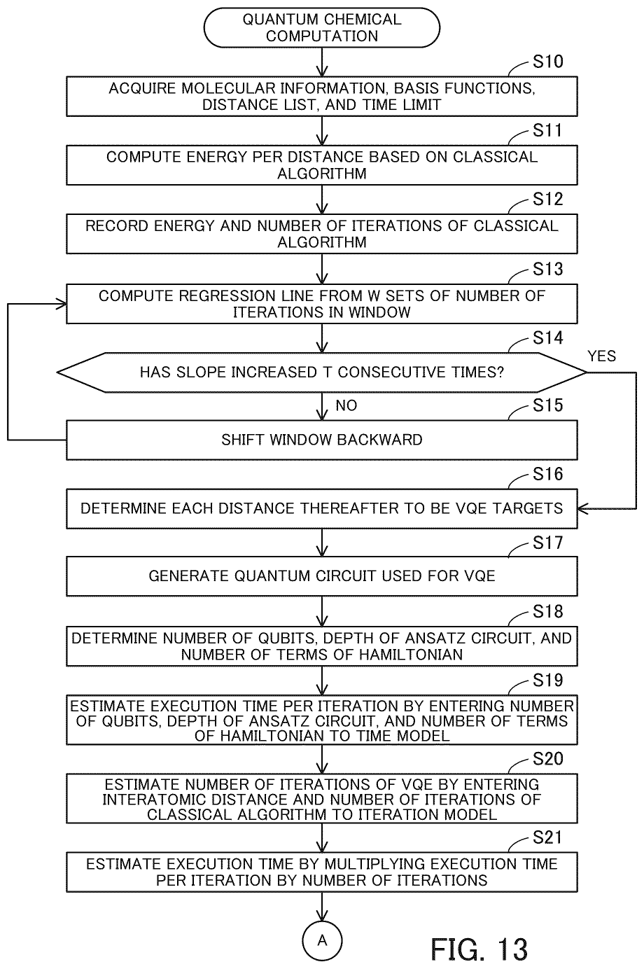

FIG. 13 is a flowchart illustrating an example of the first half of a procedure of a quantum chemical computation. (S10) The algorithm control unit 126 acquires molecular information, basis functions, a distance list, and a time limit. The distance list indicates a plurality of distances for which their respective energies are computed.

(S11) The CCSD execution unit 124 computes the energy for each distance indicated in the distance list, based on a classical algorithm such as CCSD(T). In this step, the CCSD execution unit 124 measures the numbers of iterations until their respective energies converge.

(S12) The algorithm control unit 126 records, for each distance indicated in the distance list, the energy and the number of iterations of the classical algorithm computed in step S11. The algorithm control unit 126 arranges the numbers of iterations of the classical algorithm in the ascending order of the distances. The algorithm control unit 126 creates a window having a width W in which W sets of the number of iterations are included, and sets the window on the shortest distance side. W is an integer of 2 or greater. For example, W=5.

(S13) The algorithm control unit 126 computes a regression line based on a least squares method from the W sets of the number of iterations included in the window.

(S14) The algorithm control unit 126 has calculated the regression line at least T+1 times in step S13. The algorithm control unit 126 determines whether the slope of the regression line has increased T consecutive times. T is an integer of 1 or greater. For example, T=3. If the slope has increased T consecutive times, the process proceeds to step S16. If the regression line has been calculated no more than T times or if the slope has not increased T consecutive times, the process proceeds to step S15.

(S15) The algorithm control unit 126 shifts the window in the direction in which the distance increases by one level. Next, the process returns to step S13.

(S16) The algorithm control unit 126 determines the longest distance in the window and each distance longer than this longest distance to be the VQE target distances.

(S17) The VQE execution unit 125 generates a quantum circuit used for VQE based on the molecular information, for each of the VQE target distances determined in step S16.

(S18) The algorithm control unit 126 determines the number of qubits, the depth of the ansatz circuit, and the number of terms of the Hamiltonian from the quantum circuit generated in step S17.

(S19) The algorithm control unit 126 estimates the execution time per iteration of VQE by entering the number of qubits, the depth of the ansatz circuit, and the number of terms of the Hamiltonian to a trained time model. The execution time per iteration is an estimated value obtained in view of robustness and uncertainty, for example.

(S20) The algorithm control unit 126 estimates the number of iterations of VQE by entering the interatomic distance and the number of iterations of the classical algorithm to a trained iteration model. The number of iterations is an estimated value obtained in view of robustness and uncertainty, for example.

(S21) The algorithm control unit 126 estimates the VQE execution time by multiplying the execution time per iteration estimated in step S19 by the number of iterations estimated in step S20.

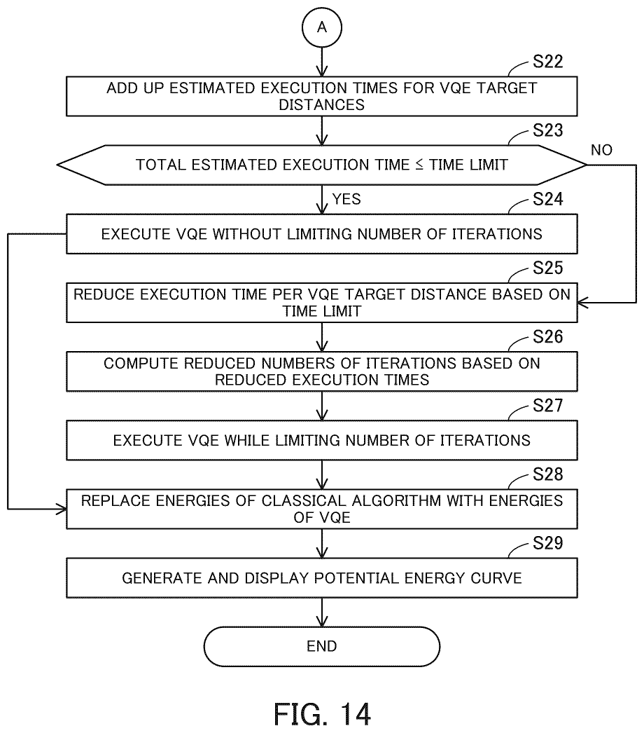

FIG. 14 is a flowchart illustrating an example of the second half of the procedure of the quantum chemical computation. (S22) The algorithm control unit 126 adds up the estimated execution times computed in step S21 for the plurality of VQE target distances.

(S23) The algorithm control unit 126 determines whether the total estimated execution time is equal to or less than the time limit. If the total estimated execution time is equal to or less than the time limit, the process proceeds to step S24. If the total estimated execution time exceeds the time limit, the process proceeds to step S25.

(S24) The VQE execution unit 125 computes the energy for each VQE target distance by executing VQE without limiting the number of iterations. The algorithm control unit 126 records the computed VQE energies. Next, the process proceeds to step S28.

(S25) Based on the time limit, the algorithm control unit 126 reduces the execution time for each VQE target distance such that the individual execution time is equal to or less than the corresponding estimated execution time. In this step, the algorithm control unit 126 reduces the total reduced execution time to be equal to or less than the time limit. The ratio of the reduced execution time with respect to the estimated execution time may be common to all the VQE target distances. Alternatively, different ratios may be used for different VQE target distances.

(S26) The algorithm control unit 126 computes the reduced number of iterations for each VQE target distance based on the reduced execution times computed in step S25. For example, the algorithm control unit 126 computes the individual reduced number of iterations such that the corresponding ratio of the reduced execution time with respect to the estimated execution time becomes the same as the corresponding ratio of the reduced number of iterations with respect to the estimated number of iterations.

(S27) The VQE execution unit 125 computes the energy for each of the plurality of VQE target distances by executing VQE such that the corresponding number of iterations does not exceed the corresponding reduced number of iterations in step S26. If the number of iteration for a VQE target distance reaches the corresponding reduced number of iterations, the VQE execution unit 125 forcibly stops VQE even if the energy has not yet converged, and outputs the finally computed energy.

(S28) Of all the distances included in the distance list, regarding the VQE target distances, the algorithm control unit 126 replaces the energies of the classical algorithm computed in step S11 by the energies of VQE computed in step S27.

(S29) The energy visualization unit 127 generates a potential energy curve from the plurality of energies corresponding to the plurality of distances obtained after the replacement in step S28. The energy visualization unit 127 displays the generated potential energy curve.

As described above, the information processing apparatus 100 according to the second embodiment executes a quantum chemical computation and generates a potential energy curve indicating the relationship between the interatomic distance and the ground-state energy of a molecule. In this way, the information processing apparatus 100 is able to provide useful information about characteristics of the molecule, and to assist research and development in material development, drug development, etc.

In addition, for all the interatomic distances, the information processing apparatus 100 computes the energy using a classical algorithm that takes a shorter execution time. The information processing apparatus 100 also computes the energy using VQE that achieves a higher accuracy, for some long interatomic distances. Thus, the information processing apparatus 100 is able to efficiently generate a potential energy curve while balancing the accuracy and the execution time.

In addition, the information processing apparatus 100 detects a boundary point at which the number of iterations of the classical algorithm sharply rises, and selects the distances after the boundary point as the VQE target distances. In this way, the interatomic distances at which the accuracy is low based on the classical algorithm are automatically determined, and the risk that preferable interatomic distances are omitted from selection targets is reduced by computing the energy based on VQE.

In addition, the information processing apparatus 100 limits the number of iterations for each of the plurality of VQE target distances such that the total VQE execution time is equal to or less than the time limit. As a result, compared with a case in which VQE is actually executed only on some VQE target distances without limiting the number of iterations, the overall accuracy of the potential energy curve is improved further. For example, it is possible to reduce the risk that an unnatural potential energy curve in which an energy of low accuracy computed by a classical algorithm and an accurate energy computed by VQE are discontinuous is generated.

In addition, the information processing apparatus 100 estimates the individual VQE execution time and number of VQE iterations by using a trained machine learning model, from features of the structure of a quantum circuit and a measured value of the number of iterations of the classical algorithm. In this way, the VQE execution time and the number of VQE iterations are accurately estimated before the execution of VQE. Thus, the information processing apparatus 100 is able to generate a potential energy curve as accurate as possible while complying with the time limit specified by the user.

In addition, the information processing apparatus 100 may set the ratio of the reduced execution time with respect to the estimated execution time to be smaller for a longer VQE target distance, and may strongly limit the number of iterations. In this way, even when the total execution time is reduced, the accuracy is little affected.

In one aspect, it is possible to efficiently compute the molecular energies corresponding to a plurality of interatomic distances.

All examples and conditional language provided herein are intended for the pedagogical purposes of aiding the reader in understanding the invention and the concepts contributed by the inventor to further the art, and are not to be construed as limitations to such specifically recited examples and conditions, nor does the organization of such examples in the specification relate to a showing of the superiority and inferiority of the invention. Although one or more embodiments of the present invention have been described in detail, it should be understood that various changes, substitutions, and alterations could be made hereto without departing from the spirit and scope of the invention.

What is claimed is:

1. A non-transitory computer-readable recording medium storing therein a computer program that causes a computer to execute a process comprising:
computing, based on molecular information about an analysis target molecule, for each of a plurality of interatomic distances, an estimated execution time of a first algorithm that computes a molecular energy by using quantum circuit data and an estimated number of iterations of an iteration process executed during the estimated execution time;
determining a limited number of iterations for each of the plurality of interatomic distances, based on a specified time limit and based on the estimated execution time and the estimated number of iterations for each of the plurality of interatomic distances; and
computing the molecular energy for each of the plurality of interatomic distances based on the limited number of iterations determined, by executing the first algorithm.

2. The non-transitory computer-readable recording medium according to claim 1, wherein the limited number of iterations determined for at least one of the plurality of interatomic distances is less than the corresponding estimated number of iterations.

3. The non-transitory computer-readable recording medium according to claim 1, wherein the determining includes determining an upper execution time limit for each of the plurality of interatomic distances, based on the time limit and a sum of the estimated execution time of each of the plurality of interatomic distances, and determining an upper limit of a number of iterations for each of the interatomic distances, based on the upper execution time limit and the estimated number of iterations.

4. The non-transitory computer-readable recording medium according to claim 1, wherein a ratio of the limited number of iteration with respect to the estimated number of iteration differs among the plurality of interatomic distances.

5. The non-transitory computer-readable recording medium according to claim 4, wherein among the plurality of interatomic distances, the ratio is set to be smaller for a longer interatomic distance.

6. The non-transitory computer-readable recording medium according to claim 1, wherein the computing of the molecular energy includes stopping the first algorithm when a number of iterations executed has reached the limited number of iterations determined, and outputting the molecular energy based on a result of the iteration process that has been executed before the stopping.

7. The non-transitory computer-readable recording medium according to claim 1, wherein the process further includes:
computing molecular energies of other interatomic distances, which are different from the plurality of interatomic distances, by executing a second algorithm different from the first algorithm, and outputting information about a potential energy curve in which the plurality of interatomic distances and the other interatomic distances are associated with the molecular energies.

8. A molecular simulation method comprising:
computing, by a processor, based on molecular information about an analysis target molecule, for each of a plurality of interatomic distances, an estimated execution time of a first algorithm that computes a molecular energy by using quantum circuit data and an estimated number of iterations of an iteration process executed during the estimated execution time;
determining, by the processor, a limited number of iterations for each of the plurality of interatomic distances, based on a specified time limit and based on the estimated execution time and the estimated number of iterations for each of the plurality of interatomic distances; and
computing, by the processor, the molecular energy for each of the plurality of interatomic distances based on the limited number of iterations determined, by executing the first algorithm.

9. An information processing apparatus comprising:
a memory configured to store molecular information about an analysis target molecule and a plurality of interatomic distances; and
a processor coupled to the memory and the processor configured to:
compute, based on the molecular information, for each of the plurality of interatomic distances, an estimated execution time of a first algorithm that computes a molecular energy by using quantum circuit data and an estimated number of iterations of an iteration process executed during the estimated execution time;
determine a limited number of iterations for each of the plurality of interatomic distances, based on a specified time limit and based on the estimated execution time and the estimated number of iterations for each of the plurality of interatomic distances; and
compute the molecular energy for each of the plurality of interatomic distances based on the limited number of iterations determined, by executing the first algorithm.
